(19) United States
(12) Patent Application Publication
(10) Pub. No.: US 2026/0009710 A1
(43) Pub. Date: Jan. 8, 2026

(54) PNEUMATIC-ELECTROMAGNETIC-DRIVEN IMPACT DEVICE FOR NONDESTRUCTIVE DETECTION OF FRUIT TEXTURE

(71) Applicants: ZHEJIANG UNIVERSITY, Zhejiang (CN); ZHEJIANG KEPLER TECHNOLOGY CO., LTD., Zhejiang (CN)

(72) Inventors: Huirong XU, Zhejiang (CN); Kun TAO, Zhejiang (CN); Yibin YING, Zhejiang (CN); Xiuqin RAO, Zhejiang (CN); Lin LI, Zhejiang (CN)

(73) Assignees: ZHEJIANG UNIVERSITY, Zhejiang (CN); ZHEJIANG KEPLER TECHNOLOGY CO., LTD., Zhejiang (CN)

(21) Appl. No.: 19/331,938

(22) Filed: Sep. 17, 2025

Related U.S. Application Data
(63) Continuation of application No. PCT/CN2024/138896, filed on Dec. 12, 2024.

(30) Foreign Application Priority Data
Dec. 22, 2023 (CN) 202311781145.5

(51) Int. Cl. G01N 3/317; G01N 33/02

(52) U.S. Cl. CPC G01N 3/317; G01N 33/025

(57) ABSTRACT

A pneumatic-electromagnetic-driven impact device for nondestructive detection of fruit texture. The bellow support member is connected to a connection plate and a bellow. A pneumatic connector is mounted on the bellow support member. A support spring is connected to the bellow support member, while the support spring is fixedly connected to the impactor housing cover. The impactor housing is connected to the impactor housing cover and the bellow. An insulating sleeve is installed inside the impactor housing, forming a hollow region between the sleeve and the housing for placing the electromagnetic coils. An iron core is arranged inside the insulating sleeve, and a sliding rod is movably connected inside the iron core. Two annular magnets are connected 10 to the sliding rod via a magnetic ring base. A sensor is embedded in a sensor base, and a flexible impact head is fixedly connected to the bottom of the sensor base.

### CROSS-REFERENCE TO RELATED APPLICATION

This application is a continuation of international application of PCT application serial no. PCT/CN2024/138896, filed on Dec. 12, 2024, which claims the priority benefit of China application no. 202311781145.5, filed on Dec. 22, 2023. The entirety of each of the above-mentioned patent applications is hereby incorporated by reference herein and made a part of this specification.

### BACKGROUND

### Technical Field

The present invention belongs to the field of agricultural machinery and particularly relates to a pneumatic-electromagnetic-driven impact device for the nondestructive detection of fruit texture.

### Description of Related Art

China is one of the largest fruit producers and consumers in the world. In 2021, the country's fruit production reached 300 million tons, with a per capita consumption of 175.27 kilograms. In recent years, with the continuous improvement in living standards, consumers' growing attention to personal health and pursuit of quality life have created greater market potential for premium fruits. In 2021, the proportion of imported fruit consumption in China rose to 2.51%, and the share has been steadily increasing.

The fruit industry covers multiple stages, including production, harvesting, sorting, packaging, transportation, storage, and processing. Among them, postharvest sorting plays a crucial role in ensuring fruit quality, extending shelf life, and enhancing market competitiveness by evaluating the weight, size, external appearance, and internal quality of fruits. Texture is one of the important indicators for assessing the internal quality and mouthfeel of fruits. It also serves as an indirect measure of ripeness and freshness. Postharvest fruit texture detection can guide the processes of harvesting, sorting, transportation, storage, and sales. Through texture evaluation, fruits can be classified into different grades or specifications to meet market demands for various textures, and fruits that are overripe, deteriorated, or excessively soft can be identified to ensure high product quality for consumers.

At present, both domestic and international solutions for postharvest fruit texture detection remain limited. These systems mainly consist of an excitation unit, a sensing module, and a signal processing unit. Depending on the excitation mode and device, fruits may exhibit free or forced vibrations. Common devices for free vibration excitation include pendulum hammers, instrumented hammers, wooden sticks, and compressed air jets. Different excitation devices often lead to different detection accuracies and may affect fruit integrity.

In addition, the response signal of the fruit is also affected by the detection method and the type of sensor used. Common sensors include microphones, accelerometers, piezoelectric sensors, and laser displacement sensors. Overall, existing fruit texture detection devices and methods suffer from low automation, limited detection accuracy, poor repeatability, narrow applicability, and a high risk of causing fruit damage.

U.S. Pat. No. 5,372,030 disclosed an instrument and method based on laser-blown air for detecting fruit texture. The device delivered a stream of pressurized air to a stationary fruit and used a laser displacement sensor to record the resulting surface deformation. A texture prediction model was then established based on the relationship between the maximum surface deformation and fruit puncture firmness. However, the device was limited to detecting soft-textured fruits, and its detection accuracy was highly affected by environmental vibrations. The repeatability of the results was poor, and it was not suitable for online sorting production lines.

U.S. Pat. No. 6,539,781B1 disclosed a device and method for measuring fruit firmness. The device struck the fruit using an electromagnetically driven plunger, and two signal sensors detected the fruit vibration to estimate fruit ripeness through modeling analysis. The use of combined signal sensors enhanced the reliability of firmness detection, but the device was sensitive to fruit shape and size. Additionally, the impact force and stroke were not easily adjustable, which increased the risk of fruit damage.

Patent No. EP0981744B1 disclosed a fruit and vegetable quality evaluation device. The device pneumatically inflated a bellow until a nose piece contacted the fruit. An internal impactor then struck the fruit under inertial force, and a piezoelectric sensor detected the vibration signal and generated output pulses. While the pneumatic actuation helped reduce fruit damage, the internal impactor's striking action relied on the motion of the bellow, introducing randomness. Moreover, the use of a passive sensor resulted in low detection sensitivity and limited measurement range.

### SUMMARY

To address the problems existing in the background art, the present invention aims to provide a pneumatic-electromagnetic-driven impact device for nondestructive detection of fruit texture.

The technical solution adopted by the present invention is as follows:

The device comprises a retractable bellow motion unit, an impactor motion unit, and an impactor housing unit. The impactor housing unit is installed inside the bellow motion unit. A flexible impact head in the impactor motion unit is vertically movable within the impactor housing unit. The bellow motion unit is connected to an external air pump, which is used to change the length of the bellow. The impactor motion unit is used to impact the fruit to be detected, and a sensor is arranged therein to detect the fruit's texture.

The bellow motion unit includes a connection plate, a pneumatic connector, a bellow support member, and a retractable bellow. The top end of the bellow support member is fixedly connected to the connection plate. The pneumatic connector is mounted on the top of the bellow support member, and the top of the bellow is sleeved onto the outer sidewall of the lower part of the bellow support member. The pneumatic connector is connected to an air pump, which applies vacuum or pressurized air into the bellow support member to control the bellow's expansion and contraction.

The impactor housing unit includes an impactor housing, a support spring, and an impactor housing cover. Both the support spring and the impactor housing cover are located inside the bellow support member. The bottom end of the support spring is fixed to the inner wall of the lower end of the bellow support member, and the top end is fixed to the bottom of the impactor housing cover. The top end of the impactor housing is fixedly connected to the inner sidewall of the bottom end of the impactor housing cover, and the bottom end of the impactor housing is in a sealed connection with the bottom of the bellow.

By adjusting the internal pressure of the bellow, the position of its bottom end can be changed, thereby driving the impactor housing and its cover to move synchronously in the vertical direction.

The impactor motion unit comprises an upper magnetic ring base, a sliding rod, an upper annular magnet, an iron core, a lower annular magnet, a sensor, a flexible impact head, a sensor base, a lower magnetic ring base, an insulating sleeve, and electromagnetic coils. The insulating sleeve is installed at the upper end inside the impactor housing, forming a hollow area with the inner wall of the housing for accommodating the electromagnetic coils. The iron core is fixedly mounted on the inner wall of the insulating sleeve, and the sliding rod is vertically movable inside the iron core. The upper and lower magnetic ring bases are connected to the top and bottom ends of the sliding rod, respectively, with the upper and lower annular magnets mounted on them. The sensor base is connected to the lower magnetic ring base, the sensor is embedded within it, and the flexible impact head is fixed at its bottom, allowing axial movement during operation.

The electromagnetic coils are connected to a control circuit via control wires. The iron core is made of magnetic material, and together with the coils forms an electromagnet. The control circuit regulates the magnetic polarity of the electromagnet to change the magnetic attraction or repulsion between the electromagnet and the annular magnets, thereby controlling the vertical motion of the sliding rod.

Multiple rectangular sliding grooves are formed along the inner wall of the bellow support member in the axial direction. The impactor housing cover is vertically slidable within these grooves.

The sensor is connected to a sensor signal acquisition card via a signal wire and is used to acquire the impact signal from the fruit after being struck, which is used to assess the fruit's texture.

The lower surface of the bellow is in contact with the fruit to be detected. Both sides of the lower surface are inclined, making the device adaptable to fruits of various shapes.

The upper magnetic ring base, sliding rod, insulating sleeve, lower magnetic ring base, sensor base, impactor housing cover, and impactor housing are all made of resilient resin material.

The flexible impact head is used to strike the fruit to be detected and is made of a pre- formulated mixture of silicone or rubber.

The bellow serves as an extension connector for the internal impactor motion unit, providing flexible alignment, noise and vibration damping, and protection against dust, moisture, oil, chemicals, UV radiation, and other environmental factors.

The clamp is ring-shaped with evenly spaced through-holes along its steel strap. The ends of the strap are secured by a fastening lock forming a closed ring. The fastening screw on the lock tightens into the through-holes to constrict the strap. The steel strap wraps tightly around a stepped annular boss on the bellow and interlocks with the barb-shaped interface to reduce gas leakage and enhance system sealing. The clamp is made of corrosion-resistant stainless steel to ensure durability in various environments.

The I-shaped structure of the insulating sleeve provides reliable support and positioning, allowing the coils to be tightly wound and evenly distributed on the sleeve.

The insulating sleeve, electromagnetic coils, and iron core together form an electromagnet. According to Ampère's circuital law, a closed loop carrying current generates a surrounding magnetic field whose strength is proportional to the current. According to Faraday's law of electromagnetic induction, a change in magnetic flux induces an electromotive force in nearby conductors. By reversing the direction of the DC, the magnetic flux changes over time, inducing a reverse current to control the magnetic field direction.

In the electromagnet, the enameled wire is wound around the insulating sleeve into solenoidal electromagnetic coils, using polyimide-coated round copper wire. Each energized coil produces a magnetic field, and since the currents are in the same direction, the magnetic fields reinforce each other. The iron core inside the insulating sleeve is made of magnetic material with high permeability and easy magnetization, which helps concentrate and amplify the magnetic field, enhancing electromagnet response speed.

Initially, the magnetic polarities of the upper and lower annular magnets are opposite. When a forward DC is applied, the polarity of the electromagnet matches that of the lower annular magnet, attracting it. During the impact phase, the control circuit reverses the current, flipping the electromagnet polarity to repel the lower annular magnet and attract the upper one, thus driving the sliding rod downward for impact. After impact, polarity is adjusted again to restore the initial state.

The impactor motion unit is installed inside the impactor housing using threaded engagement. The electromagnet is mounted at the top of the impactor housing, and the sliding rod is positioned inside it for guided motion.

The bellow motion unit, impactor motion unit, and impactor housing unit are assembled to form the complete detection system. A sealed chamber is formed between the bellow motion unit and the housing unit, which connects via the pneumatic connector to an external pump. Applying a vacuum causes the bellow to retract, and supplying compressed air makes it expand.

The present invention utilizes the impactor motion unit to apply an impact to the fruit surface, exciting its vibrational response. The sensor collects surface vibration signals, which are processed to extract feature parameters. These parameters are used to construct mathematical models correlating with the fruit's texture or maturity. The model then provides an estimation result by inferring from the extracted parameters.

This invention adopts a two-stage pneumatic-electromagnetic-driven impact detection method. First, the bellow expands under pneumatic control to position the internal impactor unit near the fruit surface. Then, electromagnetic actuation drives the impact for detection. This method allows precise control of impact force and distance, thereby addressing issues of fruit shape interference and potential damage in existing devices. It enhances detection accuracy and repeatability.

The device is mainly used for nondestructive quality detection of fruits such as watermelon, mango, kiwi, and peach. It is suitable for use in indoor smart fruit sorting lines, allowing individual inspection of fruits on a conveyor and nondestructive classification based on maturity or quality. The device offers high automation, high accuracy, stable repeatability, wide applicability, and low risk of fruit damage, providing significant potential for high-throughput, high-precision texture detection applications.

### ADVANTAGES OF THE INVENTION

The device enables postharvest nondestructive quality detection of fruits. It is suitable for indoor smart sorting lines, allowing individual inspection and maturity or quality grading based on sensor signals.

The invention features high automation, high detection accuracy, stable repeatability, wide applicability, and low risk of fruit damage. It contributes significantly to fruit grading and standardization, added value enhancement, supply chain efficiency improvement, premium fruit market development, and the overall advancement of the fruit industry.

### BRIEF DESCRIPTION OF THE DRAWINGS

FIG. 1 is an isometric view of the device according to the present invention;

FIG. 2A is an external view of the device according to the present invention;

FIG. 2B is a full sectional view of the device according to the present invention;

FIG. 3 is an exploded view of the device according to the present invention;

FIG. 4 is a full sectional view of the external bellow motion unit of the device;

FIG. 5 is an isometric full sectional view of the external bellow motion unit of the device;

FIG. 6 is an isometric view of the internal impactor motion unit of the device;

FIG. 7 is a full sectional view of the internal impactor motion unit of the device;

FIGS. 8 is a schematic view showing different working states of the electromagnet structure within the internal impactor motion unit during fruit texture detection;

FIGS. 9 is a schematic view showing different working states of the device during fruit texture detection;

FIG. 10 is an isometric view of the embodiment;

### IN THE FIGURES

1.Connection plate, 2.Pneumatic connector, 3.Signal wire, 4.Control wire, 5.Bellow support member, 6.Bellow, 7.Clamp, 8.Screw, 9.Upper magnetic ring cover, 10.Upper magnetic ring base, 11.Sliding rod, 12.Upper annular magnet, 13.Fitting iron cylinder, 14.Iron core, 15.Lower annular magnet, 16.Sensor, 17.Impactor housing, 18.Sealing rubber ring, 19.Impact head, 20.Sensor base, 21.Lower magnetic ring base, 22.Insulating sleeve, 23.Rubber gasket, 24.Electromagnetic coils, 25.Spring baffle, 26.Baffle screw, 27.Support spring, 28.Impactor housing cover, 29.Fruit, 101. Waist-shaped hole, 102.Bolt mounting hole a, 103. Wire hole a, 104.Through-hole a, 105.Bolt mounting hole b, 501.Straight pipe threaded hole, 502. Wire hole b 503.Rectangular sliding groove 504.Annular limiting flange B 505.Annular limiting groove a 601.Stepped annular boss 602.Barb-shaped interface, 603.Annular limiting groove b, 604.V-shaped fold, 605.Annular limiting groove c, 606.Annular inclined surface, 607.Cylindrical through-hole, 701.Fastening lock, 702.Clamp steel strap, 901.Internal thread b, 902.Through-hole c, 1001.Threaded hole, 1002.External thread B, 1101.Annular flange D, 1102.External thread C, 1103.Annular limiting groove e, 1104.External thread D, 1105.Annular boss, 1106.Annular step a, 1701.Annular step b, 1702.Annular step c, 1703.External thread F, 1704.Internal thread e, 1705.Annular flange C, 1901.Blind hole, 2001.Hexagonal limiting groove, 2002.Connecting through-hole, 2003.Internal thread a, 2101.External thread A, 2102.Annular limiting groove d, 2103.Internal thread c, 2104.Through-hole b, 2201.Spool support frame, 2202.Annular limiting groove f, 2203.External thread E, 2204. Wire hole c, 2801. Wire hole d, 2802.Rectangular boss, 2803.Internal thread f.

### DESCRIPTION OF THE EMBODIMENTS

Taking mango firmness detection as an example, the following describes the specific implementation of the present invention in further detail with reference to the accompanying drawings and specific embodiments. The following examples are intended to illustrate the invention but not to limit its scope.

The device comprises a retractable bellow motion unit, an impactor motion unit, and an

impactor housing unit. The impactor housing unit is installed inside the bellow motion unit. A flexible impact head (19) in the impactor motion unit is vertically movable within the impactor housing unit. The bellow motion unit is connected to an external air pump, which serves to change the vertical length of the bellow motion unit. The impactor motion unit is used to strike the fruit (29) to be detected, and a sensor (16) is provided to detect the firmness of the fruit.

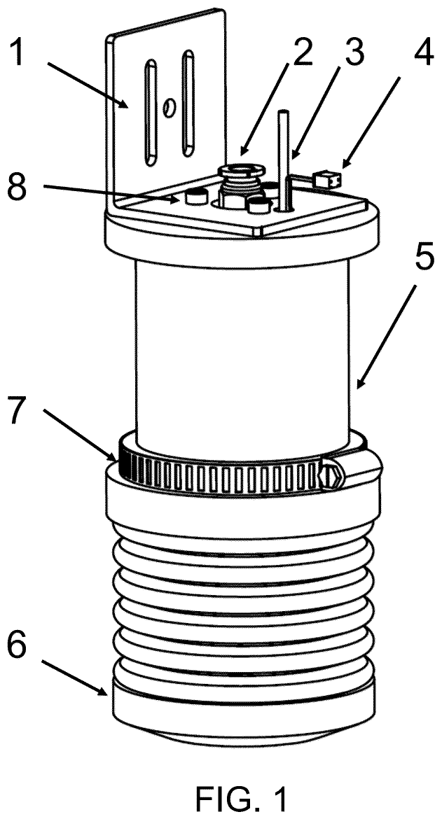

As shown in FIG. 1, the bellow motion unit comprises an L-shaped connection plate (1), a pneumatic connector (2), a bellow support member (5), and a retractable bellow (6). The top end of the bellow support member (5) is fixed to the bottom end of the connection plate (1) using screws (8). The pneumatic connector (2) is installed on the top of the bellow support member (5), and the top of the bellow (6) is sleeved over the outer sidewall of the bottom of the bellow support member (5). The pneumatic connector (2) is connected to an external air pump, which provides vacuum or pressurized air into the bellow support member (5) to adjust the expansion length of the bellow (6).

The top of the bellow (6) is clamped onto the annular flange of the bellow support member (5), and fixed in place by a clamp (7).

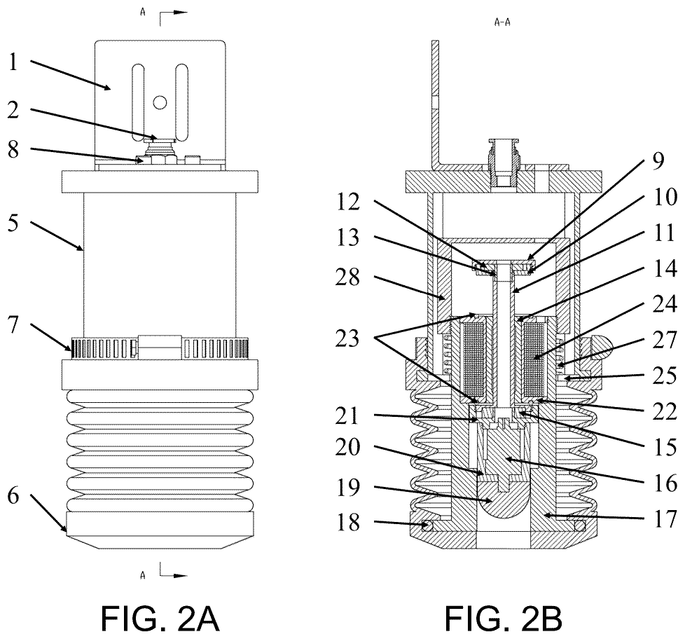

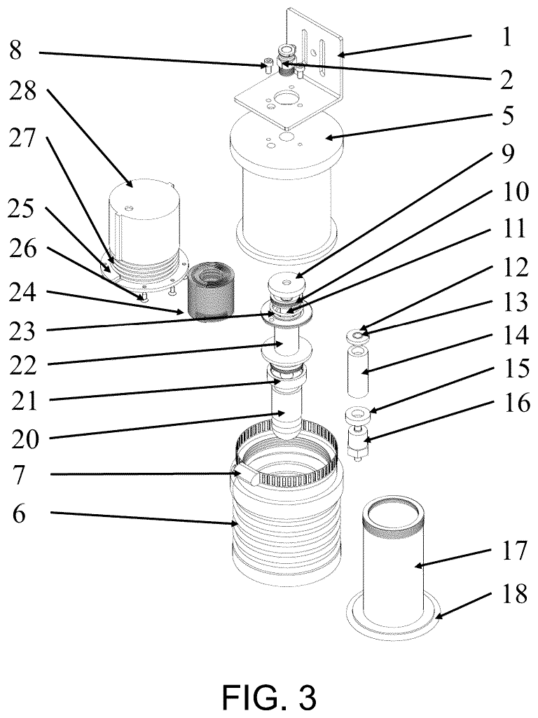

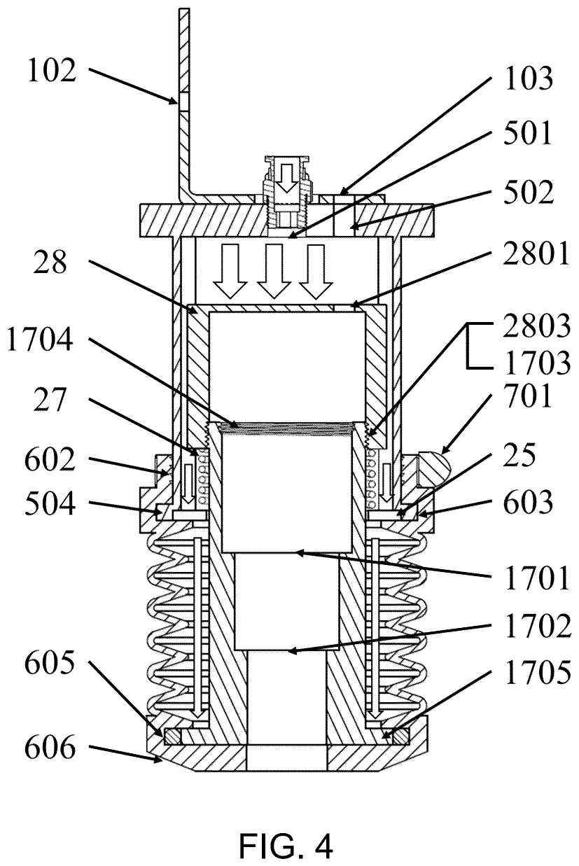

As shown in FIGS. 2A. 2B and 3, the impactor housing unit comprises the impactor housing (17), an annular support spring (27), and an annular impactor housing cover (28). The support spring (27) and impactor housing cover (28) are both located inside the bellow support member (5). The bottom of the support spring (27) is connected to the lower inner wall of the bellow support member (5) via a spring baffle (25), and its top end is coaxially fixed to the bottom of the impactor housing cover (28). The top end of the impactor housing (17) is fixedly connected to the inner sidewall of the bottom end of the impactor housing cover (28), and the bottom of the impactor housing (17) is in sealed connection with the bottom of the bellow (6) via a sealing rubber ring (18).

By varying the internal air pressure of the bellow (6), the position of its bottom end can be altered, thereby driving the impactor housing (17) and the impactor housing cover (28) to move up and down synchronously.

The lower end of the bellow (6) has an annular groove, and the upper end has an annular boss. The bottom of the impactor housing (17) is fitted into the annular groove of the bellow (6), while the bottom of the impactor housing cover (28) is supported on the annular boss at the upper end of the bellow (6) through the support spring (27) and spring baffle (25). The support spring (27) is sleeved around the outer wall of the impactor housing (17).

The impactor motion unit includes an upper magnetic ring base (10), a sliding rod (11), an upper annular magnet (12), a hollow iron core (14), a lower annular magnet (15), a sensor (16), a flexible impact head (19), a sensor base (20), a lower magnetic ring base (21), an insulating sleeve (22), and electromagnetic coils (24). The insulating sleeve (22) is installed at the upper end inside the impactor housing (17). A hollow region is formed between the outer sidewall of the insulating sleeve (22) and the inner sidewall of the impactor housing (17) for placing the electromagnetic coils (24), which are wound around the outer periphery of the insulating sleeve (22). The iron core (14) is fixedly connected to the inner sidewall of the insulating sleeve (22). The sliding rod (11), which is longer than the iron core (14), is arranged vertically movably inside the iron core (14). The upper magnetic ring base (10) and the lower magnetic ring base (21) are respectively connected to the outer walls of the upper and lower ends of the sliding rod (11). The upper annular magnet (12) and the lower annular magnet (15) are embedded in the upper and lower magnetic ring bases (10 and 21), respectively. The top end of the sensor base (20) is connected to the bottom end of the lower magnetic ring base (21). The sensor (16) is installed inside the sensor base (20), and the flexible impact head (19) is fixedly connected to the bottom of the sensor base (20), allowing the flexible impact head (19) to move up and down along its axis.

The upper magnetic ring cover (9) is mounted on the upper magnetic ring base (10), and the upper annular magnet (12) is positioned between the upper magnetic ring cover (9) and the upper magnetic ring base (10). Rubber gaskets (23) are installed at both the top and bottom junctions between the iron core (14) and the insulating sleeve (22).

The electromagnetic coils (24) are connected to a control circuit via a control wire (4). The iron core (14) is made of magnetic material, and together with the electromagnetic coils (24), forms an electromagnet. The magnetic polarity of the electromagnet is controlled by the control circuit, which regulates the magnetic attraction or repulsion between the annular magnets (12, 15) and the electromagnet, thereby controlling the vertical motion of the sliding rod (11).

Two rectangular sliding grooves (503) are provided on the inner sidewall of the bellow support member (5), and arranged axially. The sidewall of the impactor housing cover (28) is vertically slidable within these rectangular sliding grooves (503).

The sensor (16) is connected via a signal wire (3) to an external sensor signal acquisition card. The sensor (16) is used to acquire the impact signal after the fruit is struck, and the fruit's texture is then determined based on the acquired signal.

The lower surface of the bellow (6) is in contact with the fruit (29) to be detected, and both sides of the lower surface are inclined, allowing the device to accommodate fruits of various shapes.

The upper magnetic ring base (10), upper magnetic ring cover (9), spring baffle (25), sliding rod (11), insulating sleeve (22), lower magnetic ring base (21), sensor base (20), impactor housing cover (28), and impactor housing (17) are all made of resilient resin material, which offers high toughness and flexural strength, enabling them to withstand certain impact and vibration loads.

The flexible impact head (19) is used to strike the fruit (29) to be detected. It is made from a predetermined mixture of silicone or rubber.

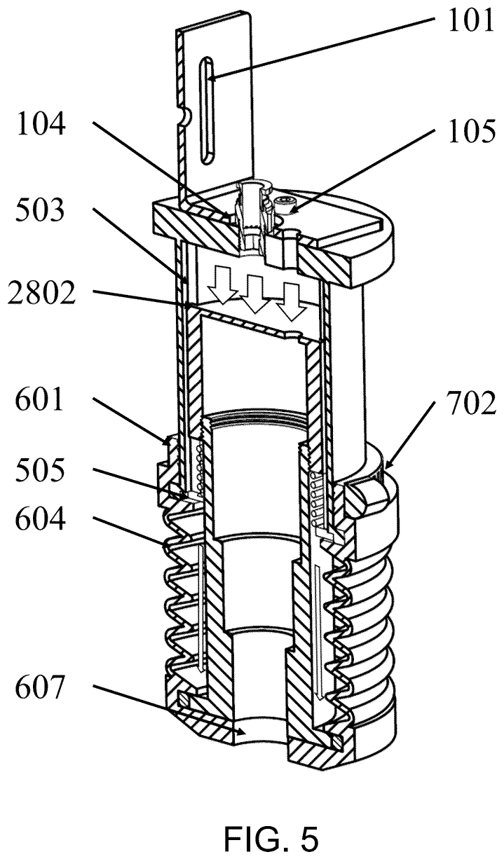

As shown in FIGS. 4 and 5, the connection plate (1) is an L-shaped metal component composed of a vertical arm and a horizontal arm, which are joined perpendicularly. The connection plate is made of high-strength stainless steel to ensure structural stability and durability. Two waist-shaped holes (101) and a bolt mounting hole a (102) are formed on the vertical arm to enable the mounting of the connection plate (1) onto a fruit sorting line. At the center of the horizontal arm lies a through-hole a (104), around which three bolt mounting holes b (105) are evenly arranged in the circumferential direction. Additionally, a wire hole a (103) is provided on the central axis, positioned in front of through-hole a (104). The three bolt mounting holes b (105), together with screws (8), are used to fix the bellow support member (5) onto the connection plate (1)

The bellow support member (5) has a hollow cylindrical body. A thick boss structure is formed at its upper end, where a straight pipe threaded hole (501), a wire hole b (502), and three threaded holes are arranged. The threaded hole (501) is coaxial with through-hole a (104) on the connection plate (1) and is used for mounting the pneumatic connector (2). The wire hole b (502) is coaxial with wire hole a (103) on the connection plate, allowing signal wire (3) and control wire (4) to be routed from inside the device and connected to a PLC (programmable logic controller) and sensor signal acquisition card. Rectangular sliding grooves (503) are formed on the left and right inner walls of the bellow support member (5) in the vertical direction. An annular limiting flange B (504) is provided at the lower end for nested installation with the bellow (6), and an annular limiting groove a (505) is provided on the lower inner wall for installing the spring baffle (25). The bellow support member (5) is made of aluminum alloy to ensure lightweight and high strength.

The bellow (6) has an open upper end, with a stepped annular boss (601) on its outer wall and a ring of barb-shaped interfaces (602) on its inner side. The upper end of the bellow (6) is clamped onto the bellow support member (5) by means of a clamp (7) with a clamp steel strap (702) locked at both ends by fastening locks (701). An annular limiting groove b (603) is also formed at the bottom of the inner wall of the stepped annular boss (601), enabling a sealed nested connection with the bellow support member (5). The main body of the bellow (6) consists of multiple V-shaped folds (604) forming a corrugated layer. The wave height, wall thickness, and spacing between folds can be designed according to application requirements. The bottom of the bellow (6) features an inverted trapezoidal end, with an annular limiting groove c (605) on the inner side for nesting the impactor housing (17) and sealing rubber ring (18), thereby forming a sealed interface. An annular inclined surface (606) is formed on the outer part of the trapezoidal end, which adjusts the planar area of the bellow's bottom through inclination angle design, making the device adaptable to fruits of various shapes. A cylindrical through-hole (607) is provided at the center of the trapezoidal end to allow the flexible impact head (19) of the impactor motion unit to move in and out for contact with the fruit (29) to be detected. Under air pressure, the bellow (6) serves as an extension connector for the impactor motion unit, contributing to alignment, noise reduction, vibration absorption, and protection. The bellow (6) is integrally formed by rubber or soft gel materials of different ratios via injection molding, and possesses a certain degree of elasticity and pressure resistance to accommodate internal pressure variations.

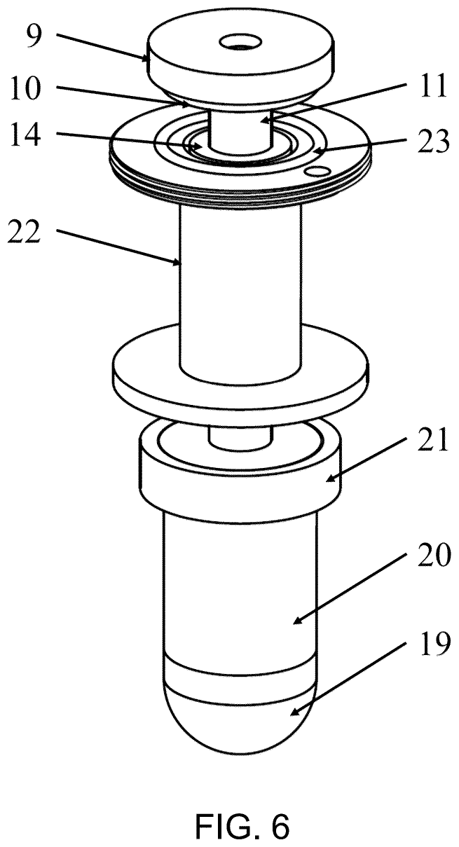

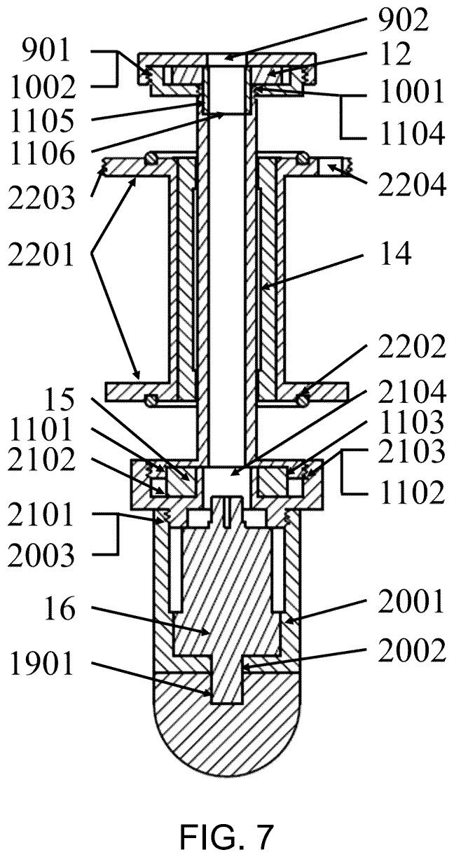

As shown in FIGS. 6 and 7, the flexible impact head (19) has a hemispherical shape and may be formed by injection molding and curing of silicone or rubber materials with various properties. A blind hole (1901) is formed at the center of the top surface of the impact head (19), into which a plastic fitting nut may be embedded. This configuration facilitates positioning and connection between the impact head (19) and the sensor (16), and allows mounting of the sensor's signal input shaft. The sensor base (20) has an open top and a closed bottom. Inside the bottom, a hexagonal limiting groove (2001) is formed, and a connection through-hole (2002) is provided at the center for sensor (16) installation. An internal thread a (2003) is provided on the inner upper end of the sensor base (20) for threaded engagement with the lower magnetic ring base (21).

The upper magnetic ring base (10) has an open top for the installation of the upper annular magnet (12). Its upper outer wall is provided with external thread B (1002) for mounting the upper magnetic ring cover (9), and a threaded hole (1001) at the center bottom for attaching the sliding rod (11). The lower magnetic ring base (21) is a cylindrical sleeve. An annular limiting groove d (2102) is formed inside its top for the lower annular magnet (15), and internal thread c (2103) is provided on the inner wall of groove d for mounting the sliding rod (11). The bottom of the lower magnetic ring base (21) forms an annular boss with external thread A (2101), which mates with internal thread a (2003) of the sensor base (20). A through-hole b (2104) is provided at the center for routing the signal wire (3) and other wires. The lower inner side of the upper magnetic ring cover (9) includes internal thread b (901) for connecting with the upper magnetic ring base (10). A through-hole c (902) is located at the top center for routing signal wire (3), etc.

The sliding rod (11) is a hollow cylindrical body. At the bottom, it has an annular flange D (1101) with external thread C (1102) on the outer surface for connection to the lower magnetic ring base (21), and an annular limiting groove e (1103) on the inner surface to constrain the lower annular magnet (15). The top of the sliding rod (11) is formed with an annular boss (1105), whose outer wall includes external thread D (1104) for connection with the upper magnetic ring base (10). An annular step a (1106) is provided inside for mounting the fitting iron sleeve (13), which adjusts the magnetic field of the upper annular magnet (12) to enhance magnetic attraction or repulsion to other magnetic components.

The iron core (14) is a hollow cylinder with an inner diameter slightly larger at both ends. Made of magnetic materials such as iron or steel, it provides excellent magnetic permeability. The sliding rod (11) is mounted inside the core's bore, and the core (14) is mounted inside the through-hole of the insulating sleeve (22). The variation in the inner diameter of the iron core helps optimize the magnetic field distribution or meet specific electromagnetic requirements, offering flexible electromagnetic tuning performance.

The impactor housing cover (28) is an inverted hollow cylindrical structure with a closed top. A wire hole d (2801) is provided at the top surface perimeter, and rectangular bosses (2802) are formed axially on both outer sidewalls. The lower end is open, and an internal thread f (2803) is formed on the inner wall at the bottom. The rectangular bosses (2802) can slide within the rectangular sliding grooves (503) of the bellow support member (5), allowing stable linear motion and accurate positioning. The wire hole d (2801) on the housing cover (28), the wire hole c (2204) on the insulating sleeve (22), wire hole a (103) on the connection plate (1), and wire hole b (502) on the bellow support member (5) are all aligned along a common central axis for routing of signal wire (3) and control wire (4) from the interior of the device.

The spring baffle (25) comprises two semi-circular halves forming a ring with an inner diameter slightly larger than the outer diameter of the main body of the impactor housing (17). Three countersunk holes are evenly distributed along the circumferential direction. The spring baffle halves are fixed to the annular limiting groove a (505) in the bellow support member (5) using baffle screws (26). The support spring (27) is elastically connected at both ends to the impactor housing cover (28) and the spring baffle (25). The spring (27) provides restoring force through its elastic properties to reset the bellow (6), reduce load and impact on the bellow, and mitigate fatigue damage.

The impactor housing (17) is a hollow cylindrical shell with two spaced annular steps b and c (1701, 1702) along its inner wall. External thread F (1703) on the upper outer surface mates with internal thread f (2803) on the lower inner side of the impactor housing cover (28). Internal thread e (1704) on the upper inner wall mates with external thread E (2203) of the insulating sleeve (22). The bottom is formed with annular flange C (1705). Step b (1701) provides a supporting surface for mounting the insulating sleeve (22), and step c (1702) offers motion space and guidance for the impactor motion unit. A sealing rubber ring (18) is embedded between the lower annular flange (1705) and the bellow (6) to fill gaps, effectively preventing fluid or gas leakage and maintaining system integrity and performance.

The insulating sleeve (22) functions as a coil spool, shaped as an I-beam in cross-section. The top and bottom annular bosses form the spool supports (2201), with external thread E (2203) on the upper boss for mounting inside the impactor housing (17). Annular limiting grooves f (2202) are formed around the top and bottom center holes. The top groove also includes wire hole c (2204). Rubber gaskets (23) are installed within the upper and lower grooves to absorb vibration and impact during striking, reducing contact and friction between mechanical parts and thereby minimizing noise and vibration transmission. The electromagnetic coils (24) are tightly and evenly wound between the two spool supports (2201) on the cylindrical wall of the sleeve, according to a defined diameter, number of turns, and layers. Both ends of the coils are welded to lead terminals with plugs, one connected to the positive power supply and the other to the negative or ground terminal.

The sensor (16) may be a piezoelectric accelerometer with an internal piezoelectric element and a built-in charge amplifier. It uses a single two-core cable for power supply and signal output, allowing direct connection to a measurement device without requiring additional amplifiers or signal conditioning units.

The impact parameters of the present invention include the material of the impact head, the impact force, and the impact distance. The magnitude of the impact force affects the degree of deformation of the fruit, the impact distance determines the contact area between the impact head and the fruit as well as the amount of transmitted impulse energy, and the material of the impact head influences the efficiency of impulse transmission and the fruit's vibrational response. The impact force can be adjusted by varying the diameter of the enameled wire in the electromagnetic coil structure and the maximum outer diameter of the insulating sleeve (22). Meanwhile, the impact distance can be tuned by modifying the height of the annular step b1701 inside the impactor housing (17), thereby adjusting the spacing between the flexible impact head (19) and the bottom surface of the bellow (6). Flexible impact heads (19) with different levels of hardness can be fabricated from silicone or rubber using different mixing ratios. By adjusting the internal structure of the impactor unit or selecting different materials for the flexible impact head (19), the device can be flexibly adapted to the nondestructive detection of fruits with different firmness, ensuring that the impact parameters are well-matched to the physical properties of the fruit. This enables accurate and reliable texture detection while minimizing potential damage to the fruit.

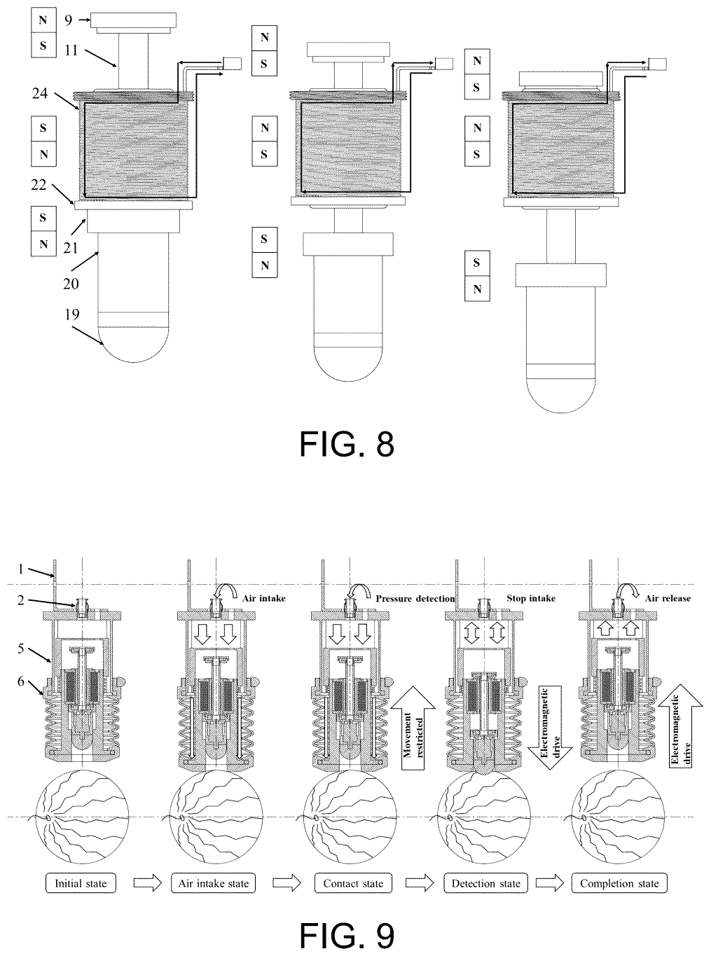

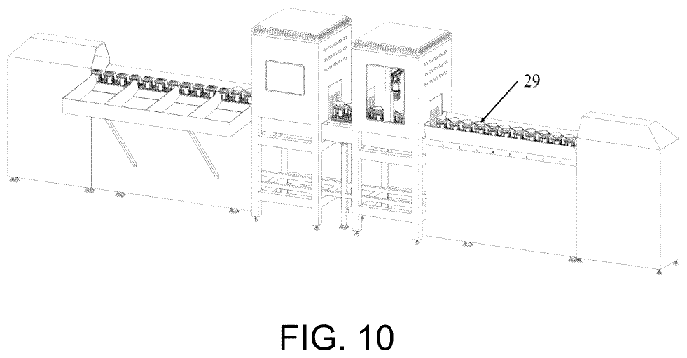

As shown in FIG. 8 to FIG. 10, the nondestructive texture detection process for fruit using a flexible impactor can be divided into five stages: initial state, air intake state, contact state, detection state, and completion state. The operational procedure of the present invention is as follows:

1. Initial state: The device is mounted on a fruit sorting line. The sensor (16) is connected

to an external sensor signal acquisition card via the signal cable (3). The electromagnetic coils (24) are connected to a driver board and a PLC (Programmable Logic Controller) via the control cable. The entire device is connected to a pneumatic control system through the pneumatic connector (2). The bellow (6) remains stationary, maintaining its original length. The impactor unit is held at the lower end of the insulating sleeve (22), attracted by the interaction between the lower annular magnet (15) and the core (14). The fruit to be detected (29) is placed on a free fruit holder on the sorting line and transported directly beneath the nondestructive detection device by a conveyor belt.

2. Air intake state: The pneumatic control system introduces pressurized air into the detection device via the pneumatic connector (2). The air flows through the upper cavity of the bellow support member (5) and the rectangular sliding grooves (503), then spreads downward into the bellow (6), which expands vertically and drives the impactor unit to approach the surface of the fruit (29).

3. Contact state: The bottom of the bellow (6) comes into contact with the surface of the fruit (29). As the pressurized air continues to flow in, the downward movement of the bellow is restricted, causing the internal pressure of the system to rise. The pressure sensor in the pneumatic control system continuously transmits analog signals to the PLC, which determines whether the system pressure has reached a predefined threshold. When the PLC detects that the pressure exceeds the threshold, it indicates that the bellow has contacted the fruit.

4. Detection state: The PLC processes the received analog signal from the pressure sensor and sends a signal to the solenoid valve in the pneumatic control system to stop air intake, while simultaneously sending a signal to the electromagnetic control circuit to initiate the impact. The sensor (16) converts the vibration signal generated by the impact into an electrical signal, which is then transmitted through the signal acquisition card to an upper computer for signal processing and texture analysis.

5. Completion state: The PLC sends a signal to the electromagnetic control circuit to reset the impactor, and another signal to the solenoid valve to activate the vacuum generator, enabling rapid restoration of the bellow (6) to its initial position.

The pneumatic control system comprises an air compressor, air source processor, 2-position 5-way solenoid valve, 2-position 2-way solenoid valve, pressure sensor, switching power supply, PLC controller, control driver module, and a vacuum generator.

The foregoing embodiments are intended solely to illustrate the present invention, and not to limit it. Any modifications or alterations made within the spirit and scope of the claims of the present invention are considered to fall within the scope of its protection.

What is claimed is:

1. A pneumatic-electromagnetic-driven impact device for nondestructive detection of a fruit texture, comprising:
a retractable bellow motion unit, an impactor motion unit, and an impactor housing unit; the impactor housing unit is installed inside the retractable bellow motion unit; a flexible impact head in the impactor motion unit is vertically movable within the impactor housing unit; the retractable bellow motion unit is connected to an external air pump, which is used to change a length of the retractable bellow motion unit; the impactor motion unit is used to strike a fruit to be detected, and a sensor is provided in the impactor motion unit for detecting a texture of the fruit.

2. The pneumatic-electromagnetic-driven impact device according to claim 1, wherein the retractable bellow motion unit comprises a connection plate, a pneumatic connector, a bellow support member, and a retractable bellow; a top end of the bellow support member is fixedly connected to the connection plate, the pneumatic connector is connected to a top of the bellow support member, and a top of the retractable bellow is sleeved onto an outer sidewall of a lower end of the bellow support member; the pneumatic connector is externally connected to an air pump, which applies vacuum or pressurized air into the bellow support member, thereby changing a length of the retractable bellow.

3. The pneumatic-electromagnetic-driven impact device according to claim 2, wherein the impactor housing unit comprises an impactor housing, a support spring, and an impactor housing cover; the support spring and the impactor housing cover are both located inside the bellow support member; a bottom end of the support spring is connected to an inner sidewall of a bottom of the bellow support member, and a top end of the support spring is fixedly connected to a bottom end of the impactor housing cover; a top end of the impactor housing is fixedly connected to an inner sidewall of the bottom end of the impactor housing cover, and a bottom end of the impactor housing is sealingly connected to a bottom of the retractable bellow; by changing an internal air pressure of the retractable bellow, a position of the bottom of the retractable bellow is changed, thereby driving the impactor housing and the impactor housing cover to move up and down synchronously.

4. The pneumatic-electromagnetic-driven impact device according to claim 3, wherein the impactor motion unit comprises an upper magnetic ring base, a sliding rod, an upper annular magnet, an iron core, a lower annular magnet, the sensor, the flexible impact head, a sensor base, a lower magnetic ring base, an insulating sleeve, and electromagnetic coils; the insulating sleeve is installed at an upper end inside the impactor housing, and a hollow region is formed between the insulating sleeve and an inner wall of the impactor housing for placing the electromagnetic coils; the iron core is fixedly connected to an inner wall of the insulating sleeve, and the sliding rod is arranged movably up and down inside the iron core; the upper magnetic ring base and the lower magnetic ring base are respectively connected to an upper end and a lower end of the sliding rod; the upper annular magnet and the lower annular magnet are mounted on respective magnetic ring bases; a top end of the sensor base is connected to a bottom end of the lower magnetic ring base, the sensor is installed inside the sensor base, and the flexible impact head is fixedly connected to a bottom of the sensor base, such that the flexible impact head can move up and down along its own axis.

5. The pneumatic-electromagnetic-driven impact device according to claim 4, wherein the electromagnetic coils are connected to a control circuit via a control wire, the iron core is made of magnetic material, and the electromagnetic coils and the iron core together form an electromagnet structure; the control circuit is used to control a polarity direction of an electromagnet, thereby adjusting an attraction status between the annular magnets and the electromagnet, and further controlling a up-and-down movement of the sliding rod.

6. The pneumatic-electromagnetic-driven impact device according to claim 4, wherein several rectangular sliding grooves are formed on the inner sidewall of the bellow support member, each of which is arranged axially along the bellow support member; the impactor housing cover is connected movably up and down within the rectangular sliding grooves.

7. The pneumatic-electromagnetic-driven impact device according to claim 4, wherein the sensor is connected to a sensor signal acquisition card via a signal wire, and the sensor is configured to collect impact signals from the fruit after being struck, thereby determining the fruit texture.

8. The pneumatic-electromagnetic-driven impact device according to claim 2, wherein a lower surface of the retractable bellow is in contact with the fruit to be tested, and both sides of the lower surface of the retractable bellow are inclined, making the pneumatic-electromagnetic-driven impact device suitable for fruits of different shapes.

9. The pneumatic-electromagnetic-driven impact device according to claim 4, wherein the upper magnetic ring base, the sliding rod, the insulating sleeve, the lower magnetic ring base, the sensor base, the impactor housing cover, and the impactor housing are all made of resilient resin material.

10. The pneumatic-electromagnetic-driven impact device according to claim 4, wherein the flexible impact head is used to strike the fruit to be tested, and is made of predetermined ratio of silicone or rubber.
